(19) United States
(12) Patent Application Publication
(10) Pub. No.: US 2006/0286049 A1
(43) Pub. Date: Dec. 21, 2006

(54) Cosmetic composition comprising a tribochromic compound, process using this composition and uses

(76) Inventors: Chrystel Grethen-Pourille, Clichy (FR); Luc Gourlaouen, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/362,757

(22) Filed: Feb. 28, 2006

Related U.S. Application Data
(60) Provisional application No. 60/694,432, filed on Jun. 28, 2005.

(30) Foreign Application Priority Data
Feb. 28, 2005 (FR) 05 02004

(51) Int. Cl. A61K 8/49

(52) U.S. Cl. 424/63

(57) ABSTRACT

The present disclosure relates to a cosmetic composition comprising, in an appropriate cosmetic medium, at least one tribochromic compound. The present disclosure also relates to a process for making up the lips, skin, or integuments using the compostions disclosed herein, wherein the at least one tribochromatic compound has the property of changing coloration or shade when, once deposited onto the integuments, the lips or the skin, it undergoes a conformational change subsequent to a mechanical stress.

This application claims benefit of U.S. Provisional Application No. 60/694,432, filed Jun. 28, 2005, the contents of which are incorporated herein by reference. This application also claims benefit of priority under 35 U.S.C. § 119 to French Patent Application No. FR 05 02004, filed Feb. 28, 2005, the contents of which are also incorporated herein by reference.

The present disclosure relates to a cosmetic composition comprising at least one tribochromic compound, and also to the processes and uses using this composition.

In the cosmetics field, in particular in the hair dyeing field and in the makeup field, there has existed, for a number of years, a need to provide dye compositions for which the coloration obtained can be changed or shaded simply.

It is known practice to dye human keratin fibers, including the hair, with dye compositions comprising oxidation dye precursors, generally called oxidation bases. These oxidation bases are colorless or weakly colored compounds which, when combined with oxidizing products, give rise to colored compounds.

It is also known that the shades obtained with these oxidation bases can be varied by combining them with coloration modifiers or couplers. The variety of molecules involved in terms of oxidation bases and couplers makes it possible to obtain a rich array of colors.

It is also known practice to dye human keratin fibers by direct dyeing, comprising applying to the keratin fibers direct dyes, which are colored and coloring molecules that have an affinity for said fibers.

The colorations obtained may be temporary or semi-permanent, i.e. they fade at best after shampooing only 4 or 5 times. These colorations can have the advantage of not resulting in chemical degradation of the keratin.

These dyeing methods can make it possible to obtain colorations that can be chromatic. However, they do not make it possible for a user to readily change coloration in a short period of time.

This is because changes in coloration in the hair-related field are generally generated by bleaching/dyeing processes using hydrogen peroxide, which is an aggressive agent for the keratin fiber. These successive changes in coloration of keratin fibers, such as the hair, can damage or sensitize said fibers. In fact, differences in coloration along the same keratin fiber, between its tip and its root, are observed on hair that has undergone several successive colorations, and this is then referred to as selectivity.

Similarly, in the makeup field, the use of colored or coloring products for making the face, or even other parts of the body, more attractive or for masking imperfections of the skin is very widespread, for instance the use of blusher, of foundation, or of powder for covering the skin, or the use of mascara, lipstick, nail varnish, or pencils for making up the skin, the lips or the nails.

The color provided by these products (foundation or lipstick) may not be modified while in use. In fact, in order to obtain a change in color, it is necessary to remove the makeup and then reapply makeup using products in the desired shades.

At this time, no dyeing or makeup product exists that makes it possible, using an existing colored or coloring product, to change its color or its shade rapidly and easily.

Consequently, there remains a need for substances having the property of changing color or shade uniformly or nonuniformly, and rapidly and easily, without attacking the material to be “recolored”.

It has now been discovered, surprisingly, that it is possible to solve this problem using a cosmetic composition comprising at least one tribochromic compound.

Tribochromic compounds are colored compounds that have the property of changing color through simple mechanical stress, such as the force of manual rubbing. When this compound is impregnated or applied onto a support, the color of the support will undergo a color change at the time of the mechanical stress.

This property is known in the field of inks that cannot be forged. International Application Publication No. WO 94/26729 (University College Cardiff Consultants Limited) describes the synthesis and the use of furan derivatives in the field of inks that cannot be forged.

Pyrazine derivatives are described in International Application Publication No. WO 03/028684. This document discloses their use as hair dyes. Similarly, pyrazine derivatives are disclosed in European Patent EP 579 835 wherein their method of synthesis is described along with their use for the manufacture of films, which will be used in the construction of greenhouses, in order to modify the wavelength of the light (natural or artificial), thus making it possible to promote plant growth. Neither of these two applications mentions specific compounds that are tribochromic in nature.

Other subjects, aspects, characteristics and benefits of the present disclosure will emerge more clearly on reading the description and the examples which follow.

The present disclosure relates to a cosmetic composition comprising, in an appropriate cosmetic medium, at least one tribochromic compound.

Without wishing to be bound to any theory, it is believed that the mechanical stress having the force of rubbing or of manual friction induces a conformational change in the tribochromic compound, which brings about the change in color.

For example, the forces to be applied in order to obtain the desired tribochromic effect range from 0.01 N to 50 N, for instance from 0.1 N to 20 N, such as from 1 N to 20 N.

This color change is irreversible. Once the conformation of the molecule has been modified, the latter cannot return to its initial conformation.

The color shade obtained depends on the force and the time of the mechanical stress exerted on the material to be recolored. The longer and the greater the mechanical stress, the greater the amount of tribochromic compounds whose conformation will have changed and the greater the modification of the color or the shade. Thus, it is necessary to mechanically stress the material to be recolored until the desired shade is obtained.

The user may thus modify the color evenly or modify only certain parts of the area colored by the tribochromic compound.

In the hair-related field, the color change can be obtained, for example, subsequent to brushing the lock, or the entire head of hair.

For nail varnishes, the color change can be obtained by the action of the fingers or the nails or of a suitable object.

When the composition is applied to the skin, the color change can be obtained by rubbing the skin with the fingers, a brush or a “blush”.

For example, the at least one tribochromic compound that can be used in the compositions according to the present disclosure can be chosen from those of formulae (I) to (III):

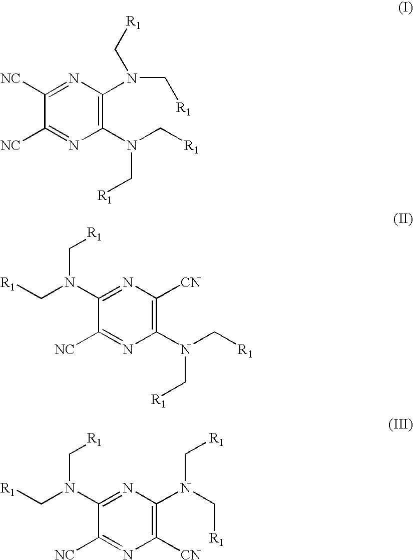

wherein

R₁, which may be identical or different, is chosen from condensed and noncondensed C₆-C₃₀ aryl radicals optionally substituted with at least one entity chosen from halogen atoms, such as F, Cl, I or Br, and C₁-C₁₀ alkyl, hydroxyl, (C₁-C₁₀)alkoxyamino, (C₁-C₁₀)mono and dialkylamino, mono and dihydroxy(C₁-C₁₀)alkylamino, (C₁-C₁₀)alkylhydroxy(C₁-C₁₀)alkylamino, mono and polyhydroxy(C₁-C₁₀)alkyl, C₆-C₃₀ aryl, carboxyl, (C₁-C₁₀)alkoxycarbonyl, sulpho, C₂-C₁₀ acyl, (C₁-C₁₀) acyloxy, aminocarbonyl, nitro, cyano and ureido groups;

it being possible for R₁ to be substituted with an electron-withdrawing group or with an electron-donor group; for instance, the substituent R₁ can be chosen from trimethylphenyl, biphenyl and naphthalenylmethyl radicals, chlorophenyl and bromophenyl radicals.

The term “electron-withdrawing group” is understood to mean a halogen atom, a nitro group, a cyano group or an —SO₃— group.

The term “electron-donor group” is understood to mean an alkyl group; an alkoxy group; an unsubstituted amine or an amine substituted with an alkyl group; or an aryl group.

In one embodiment, the substituents R₁ are identical.

In another embodiment, the at least one tribochromic compound is chosen from those of formulae (IV) to (VII):

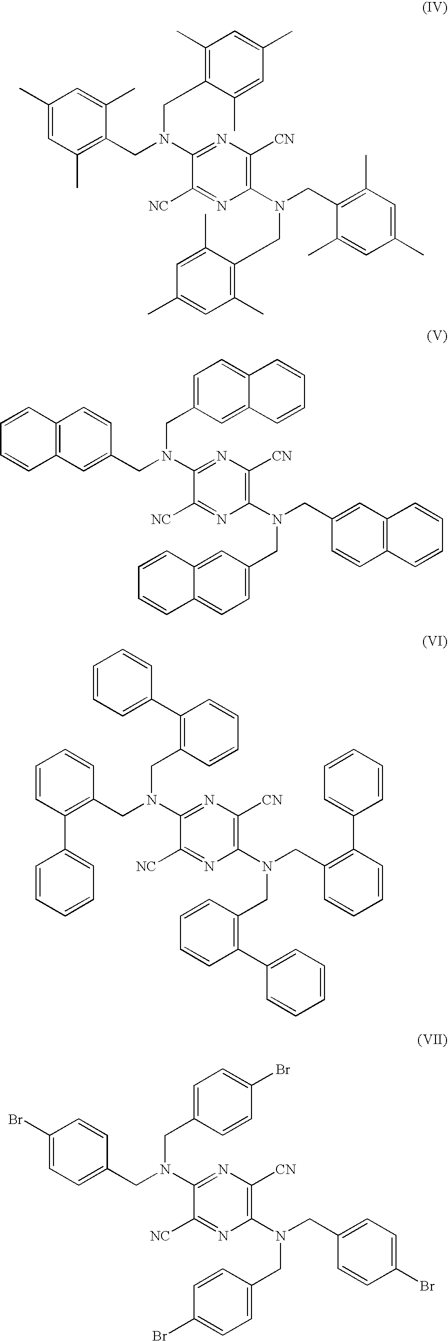

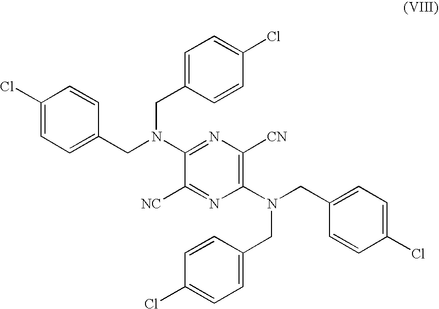

The composition of the of the present disclosure comprises from 0.0001% to 30%, such as from 0.01% to 10%, by weight of the tribochromic compound relative to the total weight of the composition.

According to still another embodiment, the composition in accordance with the present disclosure comprises, in addition to the at least one tribochromic compound, at least one adjuvant chosen from direct dyes, oxidation dyes, surfactants, thickeners, oils, waxes, gums, pigments and pearlescent agents.

The at least one additional direct dyes may be chosen from dyes that are non-ionic, cationic and anionic in nature, and may, for example, be chosen from the following red or orangey benzene dyes:
1-hydroxy-3-nitro-4-N-(γ-hydroxypropyl)aminobenzene,
N-(β-hydroxyethyl)amino-3-nitro-4-aminobenzene,
1-amino-3-methyl-4-N-(β-hydroxyethyl)amino-6-nitrobenzene,
1-hydroxy-3-nitro-4-N-(β-hydroxyethyl)aminobenzene,
1,4-diamino-2-nitrobenzene,
1-amino-2-nitro-4-methylaminobenzene,
N-(β-hydroxyethyl)-2-nitroparaphenylenediamine,
1-amino-2-nitro-4-(β-hydroxyethyl)amino-5-chlorobenzene,
2-nitro-4-aminodiphenylamine, and
1-amino-3-nitro-6-hydroxybenzene.
1-(β-aminoethyl)amino-2-nitro-4-(β-hydroxyethyloxy)benzene,
1-(β,γ-dihydroxypropyl)oxy-3-nitro-4-(β-hydroxyethyl)aminobenzene,
1-hydroxy-3-nitro-4-aminobenzene,
1-hydroxy-2-amino-4,6-dinitrobenzene,
1-methoxy-3-nitro-4-(β-hydroxyethyl)aminobenzene,
2-nitro-4′-hydroxydiphenylamine, and
1-amino-2-nitro-4-hydroxy-5-methylbenzene.

The composition in accordance with the disclosure can also comprise, in addition to, or as a replacement for, these benzene dyes, at least one additional direct dye chosen from yellow, yellow-green, blue and violet benzene dyes, azo dyes, anthraquinone, naphthoquinone and benzoquinone dyes, indigoid dyes, and triarylmethane-derived dyes.

Among these additional direct dyes, basic dyes can be used, among which non-limiting mention may be made of the dyes known in the Color Index, 3rd edition, under the names “Basic Brown 16”, “Basic Brown 17”, “Basic Yellow 57”, “Basic Red 76”, “Basic Violet 10”, “Basic Blue 26” and “Basic Blue 99”, or acidic direct dyes, among which further non-limiting mention may be made of the dyes known in the Color Index, 3rd edition, under the names “Acid Orange 7”, “Acid Orange 24”, “Acid Yellow 36”, “Acid Red 33”, “Acid Red 184”, “Acid Black 2”, “Acid Violet 43”, and “Acid Blue 62”, and cationic direct dyes such as those described in International Patent Application Nos. WO 95/01772 and WO 95/15144, and European Patent No. EP-A-0 714 954, the content of which is incorporated by reference into the present disclosure.

Among the additional yellow and yellow-green benzene direct dyes, non-limiting mention may, for example, be made of the compounds chosen from:
1-β-hydroxyethyloxy-3-methylamino-4-nitrobenzene,
1-methylamino-2-nitro-5-(β,γ-dihydroxypropyl)oxybenzene,
1-(β-hydroxyethyl)amino-2-methoxy-4-nitrobenzene,
1-(β-aminoethyl)amino-2-nitro-5-methoxybenzene,
1,3-di(β-hydroxyethyl)amino-4-nitro-6-chlorobenzene,
1-amino-2-nitro-6-methylbenzene,
1-(β-hydroxyethyl)amino-2-hydroxy-4-nitrobenzene,
N-(β-hydroxyethyl)-2-nitro-4-trifluoromethylaniline,
4-(β-hydroxyethyl)amino-3-nitrobenzenesulfonic acid,
4-ethylamino-3-nitrobenzoic acid,
4-(β-hydroxyethyl)amino-3-nitrochlorobenzene,
4-(β-hydroxyethyl)amino-3-nitromethylbenzene,
4-(β,γ-dihydroxypropyl)amino-3-nitrotrifluoromethylbenzene,
1-(β-ureidoethyl)amino-4-nitrobenzene,
1,3-diamino-4-nitrobenzene,
1-hydroxy-2-amino-5-nitrobenzene,
1-amino-2-[tris(hydroxymethyl)methyl]amino-5-nitrobenzene,
1-(β-hydroxyethyl)amino-2-nitrobenzene, and
4-(β-hydroxyethyl)amino-3-nitrobenzamide.

Among the additional blue or violet benzene direct dyes, non-limiting mention may, for example, be made of the compounds chosen from:
1-(β-hydroxyethyl)amino-4-N,N-bis(β-hydroxyethyl)amino-2-nitrobenzene,
1-(γ-hydroxypropyl)amino-4-N,N-bis(β-hydroxyethyl)amino-2-nitrobenzene,
1-(β-hydroxyethyl)amino-4-(N-methyl-N-βhydroxyethyl)amino-2-nitrobenzene,
1-(β-hydroxyethyl)amino-4-(N-ethyl-N-β-hydroxyethyl)amino-2-nitrobenzene,
1-(β,γ-dihydroxypropyl)amino-4-(N-ethyl-N-β-hydroxyethyl)amino-2-nitrobenzene,
the 2-nitro-para-phenylenediamines of formula (IV) below:

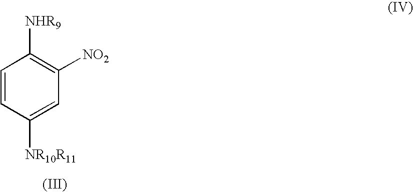

wherein:

R₁₀ is chosen from C₁-C₄ alkyl radicals, and β-hydroxyethyl, β-hydroxypropyl and γ-hydroxypropyl radicals;

R₉ and R₁₁, which may be identical or different, are chosen from β-hydroxyethyl, β-hydroxypropyl, y-hydroxypropyl and β,γ-dihydroxypropyl radicals, at least one of the radicals R₁₀, R₁₁ and R₉ being a γ-hydroxypropyl radical, and it not being possible for R₁₀ and R₁₁ to simultaneously denote a β-hydroxyethyl radical when R₉ is a γ-hydroxypropyl radical,

such as those described in French Patent No. 2 692 572.

When they are present, the additional direct dye(s) can be present in an amount ranging from 0.0005% to 12% by weight of the total weight of the composition, such as from 0.005% to 6% by weight, relative to the total weight of the composition.

The composition of the present disclosure can also comprise at least one oxidation base and/or at least one coupler conventionally used for oxidation dyeing.

By way of non-limiting example of oxidation bases, mention may be made of para-phenylenediamines, bisphenylalkylenediamines, para-aminophenols, ortho-aminophenols, aheterocyclic bases, and addition salts thereof.

The couplers can be, for example, meta-phenylenediamine couplers, meta-aminophenol couplers, meta-diphenol couplers, naphthalene couplers, heterocyclic couplers, and addition salts thereof.

When they are present, the oxidation base(s) and the coupler(s) can each be present in an amount ranging from 0.001% to 10% by weight of the total weight of the dye composition, such as from 0.005% to 6%.

When the composition according to the present disclosure comprises at least one oxidation base and/or at least one coupler, the composition can also comprise at least one oxidizing agent. The oxidizing agents conventionally used for oxidation dyeing are, for example, a chemical oxidant, such as hydrogen peroxide, urea peroxide, alkali metal bromates, persalts such as perborates and persulphates, and peracids. A biocatalytic oxidant can also be used, such as oxydase enzymes, among which mention may be made of peroxydases, 2-electron oxidoreductases such as uricases, and 4-electron oxygenases such as laccases. This enzyme generates in situ the oxidant required for the oxidation of the dye precursor, from an appropriate substrate and atmospheric oxygen.

The cosmetic compositions of the present disclosure can also comprise, as indicated above, oils, gums and/or waxes.

The cosmetically acceptable oils, which are fatty substances that are liquid at ambient temperature, can be hydrocarbon-based and/or silicone and/or fluoro oils. They can be of animal, plant, mineral or synthetic origin.

Non-limiting mention can be made of, alone or as a mixture:

hydrocarbon-based oils of animal origin, such as perhydrosqualene,

hydrocarbon-based plant oils such as sunflower oil, corn oil, soybean oil, marrow oil, grape seed oil, groundnut oil, sweet almond oil, beauty-leaf oil, palm oil, sesame oil, hazelnut oil, apricot oil, macadamia oil, castor oil, avocado oil, jojoba oil and shea butter oil, liquid triglycerides of C₄-C₁₀ fatty acids, such as heptanoic or octanoic acid triglycerides, or caprylic/capric acid triglycerides such as those sold by the company Stearineries Dubois or those sold under the names Miglyol® 810, 812 and 818 by the company Dynamit Nobel,

synthetic esters, for instance: fatty acid esters, for instance the oils of formula R3COOR4 in which R3 is chosen from higher fatty acid residues containing from 7 to 29 carbon atoms and R4 is chosen from hydrocarbon-based chains containing from 3 to 30 carbon atoms, such as, for example, isopropyl myristate, 2-ethylhexyl palmitate, 2-octyidodecyl stearate, 2-octyidodecyl erucate and isostearyl isostearate; hydroxylated esters, such as isostearyl lactate, octyl hydroxystearate, octyidodecyl hydroxystearate, diisostearyl malate and triisocetyl citrate, polyol esters, for instance propylene glycol dioctanoate, neopentyl glycol diheptanoate, diethylene glycol diisononanoate, and pentaerythritol esters,

fatty alcohols containing from 12 to 26 carbon atoms, for instance octyidodecanol, 2-butyloctanol, 2-hexyldecanol, 2-undecylpentadecanol and oleyl alcohol,

partially fluorinated and/or silicone-based hydrocarbon-based oils,

silicone oils, such as volatile or non-volatile, linear or cyclic polydimethylsiloxanes, alkyl dimethicones, silicones modified with aliphatic and/or aromatic groups, that are optionally fluorinated, or with functional groups such as hydroxyl, thiol and/or amine groups, phenylsilicone oils such as polyphenylmethylsiloxanes or phenyl trimethicones.

The oils used may be volatile and/or non-volatile. The term “Volatile oil” is understood to mean an oil capable of evaporating, at ambient temperature, from a support to which it has been applied, in other words, an oil having a measurable vapour pressure at 25° C. of greater than 0 Pa, such as ranging from 0.13 Pa to 40,000 Pa. Non-limiting mention may be made of, for example, volatile silicone oils, such as cyclic or linear volatile silicones, and cyclocopolymers. Non-limiting mention may also be made of hydrocarbon-based volatile oils, such as isoparaffins, and volatile fluoro oils.

Among the cosmetically acceptable gums and/or waxes that can be used, non-limiting mention may be made of:

silicone gums,

waxes of animal, plant, mineral or synthetic origin, such as microcrystalline waxes, paraffin, petrolatum, petroleum jelly, ozokerite, lignite wax, beeswax, lanoline and its derivatives, candellila wax, ouricury wax, carnauba wax, Japan wax, cocoa butter, cork fiber wax, sugarcane wax, hydrogenated oils that are solid at 25° C., fatty esters and glycerides that are solid at ambient temperature, polyethylene waxes and waxes obtained by Fischer-Tropsch synthesis,

silicone waxes, and

fluoro waxes.

The cosmetic compositions of the present disclosure can also comprise at least one thickener, at least one film-forming polymer, and/or at least one plasticizer.

A particulate phase comprising pigments and/or pearlescent agents can also be present in the cosmetic compositions of the present disclosure.

The term “pigments” should be understood to mean mineral or organic, white or colored particles intended to color or opacify the composition. Non-limiting mention may, for example, be made of titanium dioxide, zirconium dioxide, cerium dioxide, zinc oxide, iron oxide or chromium oxide, ferric blue, chromium hydrate, carbon black, ultramarines (aluminosilicate polysulphides), manganese pyrophosphate and certain metal powders such as silver or aluminium powders. Non-limting mention may also be made of certain lakes, such as calcium, barium, aluminium or zirconium salts. These pigments can be present in an amount ranging from 0 to 15% by weight, such as from 8% to 10% of the final composition.

The term “pearlescent agents” should be understood to mean iridescent particles which reflect light. Non-limiting mention may, for example, be made of natural mother-of-pearl, mica coated with titanium oxide, with iron oxide, with natural pigments or with bismuth oxychloride, and also colored titanium mica.

The pearlescent agents can be present in an amount ranging from 0 to 20% by weight, such as from 8% to 15% by weight, of the final cosmetic composition.

The compositions according to the disclosure can also comprise at least one thickener and/or at least one surfactant. The at least one thickener may be of mineral origin (silica) or organic origin. The organic thickeners, also called “rheology modifiers”, in one embodiment is polymeric.

The rheology modifiers may be chosen from fatty acid amides (coconut monoethanolamide or diethanolamide, oxyethylenated carboxylic acid alkyl ether monoethanolamide), cellulose-based thickeners (hydroxyethylcellulose, hydroxypropylcellulose or carboxymethylcellulose), guar gum and its derivatives (hydroxypropyl guar), gums of microbial origin (xanthan gum or scleroglucan gum), crosslinked homopolymers of acrylic acid or of acrylamidopropanesulphonic acid and associative polymers as described below.

The at least one thickener, when present, can be present in the dye composition according to the disclosure in an amount ranging from 0.01% to 10%, such as from 0.1% to 5%, of the total weight of the composition.

The associative polymers that can be used according to the present disclosure are water-soluble polymers capable, in an aqueous medium, of reversibly associating with one another or with other molecules.

Their chemical structure comprises hydrophilic regions and hydrophobic regions characterized by at least one fatty chain.

The associative polymers that can be used according to the invention may be of anionic, cationic, amphoteric and non-ionic type. In one embodiment, a non-ionic associative polymer is used.

Among associative polymers of anionic type, non-limiting mention may be made of:

(I) those comprising at least one hydrophilic unit and at least one fatty-chain allyl ether unit, for instance those whose hydrophilic unit consists of an ethylenic unsaturated anionic monomer, such as a vinyl carboxylic acid, or an acrylic acid or a methacrylic acid, or mixtures thereof, and whose fatty-chain allyl ether unit corresponds to the monomer of formula (XV) below:
CH₂═C R′CH₂O Bn R (XV)

in which R′ is chosen from H and CH₃, B is an ethyleneoxy radical, n is an integer ranging from 0 to 100, and R is chosen from hydrocarbon-based radicals chosen from alkyl, arylalkyl, aryl, alkylaryl and cycloalkyl radicals comprising from 8 to 30 carbon atoms, such as from 10 to 24, for instance from 12 to 18 carbon atoms. In one embodiment, for example, a unit of formula (XV) that can be used is a unit in which R′ is H, n is equal to 10, and R is a stearyl (C₁₈) radical.

Anionic associative polymers of this type are described and prepared, according to an emulsion polymerization process, in European Patent No. EP-0 216 479.

Among these anionic associative polymers that can be used according to the invention, non-limiting mention may be made of polymers formed from 20 to 60% by weight of acrylic acid and/or of methacrylic acid, from 5 to 60% by weight of lower alkyl (meth)acrylates, from 2 to 50% by weight of fatty-chain allyl ether of formula (XV), and of 0 to 1% by weight of a crosslinking agent which is a well known copolymerizable polyethylenic unsaturated monomer, such as diallyl phthalate, allyl (meth)acrylate, divinylbenzene, (poly)ethylene glycol dimethacrylate and methylenebisacrylamide.

Among the latter, for example, mention can be made of crosslinked terpolymers of methacrylic acid, of ethyl acrylate, of polyethylene glycol (10 EO) stearyl alcohol ether (Steareth 10), such as those sold by the company Allied Colloids under the names Salcare SC80® and Salcare SC90®, which are aqueous 30% emulsions of a crosslinked terpolymer of methacrylic acid, of ethyl acrylate and of steareth-10 allyl ether (40/50/10);

(II) those comprising at least one hydrophilic unit of olefinic unsaturated carboxylic acid type and at least one hydrophobic unit of unsaturated carboxylic acid (C₁₀-C₃₀) alkyl ester type.

For example, these polymers can be chosen from those whose hydrophilic unit of olefinic unsaturated carboxylic acid type corresponds to the monomer of formula (XVI):

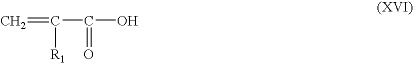

in which R₁ is chosen from H, CH₃, and C₂H₅, (i.e. acrylic acid, methacrylic acid or ethacrylic acid units), and whose hydrophobic unit of unsaturated carboxylic acid (C₁₀-C₃₀) alkyl ester type corresponds to the monomer of formula (XVII) below

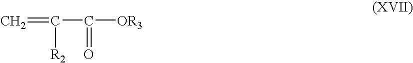

in which R₂ is chosen from H, CH₃, and C₂H₅ (i.e. acrylate, methacrylate or ethacrylate units), for example H (acrylate units) or, in one embodiment, CH₃ (methacrylate units) R₃ being chosen from C₁₀-C₃₀, such as C₁₂-C₂₂, alkyl radicals.

Unsaturated carboxylic acid (C₁₀-C₃₀) alkyl esters in accordance with the disclosure comprise, for example, lauryl acrylate, stearyl acrylate, decyl acrylate, isodecyl acrylate, dodecyl acrylate, and the corresponding methacrylates, lauryl methacrylate, stearyl methacrylate, decyl methacrylate, isodecyl methacrylate and dodecyl methacrylate.

Anionic polymers of this type are, for example, described in and prepared according to U.S. Pat. Nos. 3,915,921 and 4,509,949.

Among anionic associative polymers of this type, non-limiting mention can be made of polymers formed from a mixture of monomers comprising:

(i) essentially acrylic acid,

(ii) an ester of formula (XVII) described above and in which R₂ is chosen from H and CH₃, R₃ being an alkyl radical having from 12 to 22 carbon atoms, and

(iii) a crosslinking agent, which is a well known copolymerizable polyethylenic unsaturated monomer, such as diallyl phthalate, allyl (meth)acrylate, divinylbenzene, polyethylene glycol dimethacrylate and methylenebisacrylamide.

Among anionic associative polymers of this type, mention can be made of those consisting of 95 to 60% by weight of acrylic acid (hydrophilic unit), 4 to 40% by weight of C₁₀-C₃₀ alkyl acrylate (hydrophobic unit), and 0 to 6% by weight of crosslinking polymerizable monomer, or alternatively those consisting of 98 to 96% by weight of acrylic acid (hydrophilic unit), 1 to 4% by weight of C₁₀-C₃₀ alkyl acrylate (hydrophobic unit), and 0.1 to 0.6% by weight of crosslinking polymerizable monomer, such as those described above.

Among said polymers above, further mention can be made of the products sold by the company Goodrich under the trade names Pemulen TR1®, Pemulen TR2® and Carbopol 1382®, such as Pemulen TR1, and the product sold by the company S.E.P.P.I.C. under the name Coatex SX®;

(III) terpolymers of maleic anhydride/C₃₀-C₃₈ α-olefin/alkyl maleate, such as the product (maleic anhydride/C₃₀-C₃₈ α-olefin/isopropyl maleate copolymer) sold under the name Performa V 1608® by the company Newphase Technologies;

(IV) acrylic terpolymers comprising:

(a) approximately 20% to 70% by weight of an α,β-monoethylenically unsaturated carboxylic acid,

(b) approximately 20 to 80% by weight of a non-surfactant α,β-monoethylenically unsaturated monomer other than (a),

(c) approximately 0.5 to 60% by weight of a non-ionic monourethane which is the product of the reaction of a monohydric surfactant with a monoethylenically unsaturated monoisocyanate,

such as those described in European Patent Application EP-A-0173109, and for instance that described in Example 3, i.e. a terpolymer of methacrylic acid/methyl acrylate/ethoxylated (40 EO) behenyl alcohol dimethyl meta-isopropenyl benzyl isocyanate, as an aqueous dispersion at 25%;

(V) copolymers comprising, among their monomers, an α,β-monoethylenically unsaturated carboxylic acid and an ester of an α,β-monoethylenically unsaturated carboxylic acid and an oxyalkylenated fatty alcohol.

For example, these compounds also comprise, as monomer, an ester of an α,β-monoethylenically unsaturated carboxylic acid and a C₁-C₄ alcohol.

By way of example of this type of compound, non-limiting mention may be made of Aculyn 22® sold by the company Rohm & Haas, which is a methacrylic acid/ethyl acrylate/oxyalkylenated stearyl methacrylate terpolymer.

Among associative polymers of cationic type, non-limiting mention may be made of:

(I) cationic associative polyurethanes whose family was described by the French Patent Application No. 00/09609; it can be represented by general formula (XVIII) below:
R—X—(P)n-[L-(Y)m]r-L′-(P′)p—X′—R′ (XVIII)

in which:

R and R′, which may be identical or different, are chosen from hydrophobic groups and a hydrogen atom;

X and X′, which may be identical or different, are chosen from groups comprising an amine functional groups possibly carrying a hydrophobic group, or else the group L″;

L, L′ and L″, which may be identical or different, are chosen from groups derived from a diisocyanate;

P and P′, which may be identical or different, are chosen from groups comprising an amine functional group possibly carrying a hydrophobic group;

Y is a hydrophilic group;

r is an integer ranging from 1 to 100, such as from 1 to 50, for instance from 1 to 25;

n, m and p, each independently of the others, range from 0 to 1000;

the molecule containing at least one protonated or quaternized amine functional group and at least one hydrophobic group.

In one embodiment of these polyurethanes, the only hydrophobic groups are the groups R and R′ at the chain ends.

Another family of cationic associative polyurethanes that may be used are those of formula (XVIII) described above and in which:

R and R′ both independently represent a hydrophobic group,

X and X′ each represent a group L″,

n and p range from 1 to 1000, and

L, L′, L″, P, P′, Y and m have the meaning indicated above.

In another embodiment the cationic associative polyurethanes can be those of formula (XVIII) above in which:

R and R′ both independently represent a hydrophobic group, X and X′ each represent a group L″, n and p are 0, and L, L′, L″, Y and m have the meaning indicated above.

The fact that n and p are 0 means that these polymers do not comprise any units derived from a monomer containing an amine functional group, incorporated into the polymer during the polycondensation. The protonated amine functional groups of these polyurethanes result from the hydrolysis of isocyanate functional groups, in excess, at the end of the chain, followed by alkylation of the primary amine functional groups formed by alkylating agents containing a hydrophobic group, i.e. compounds of RQ or R′Q type in which R and R′ are as defined above and Q denotes a leaving group such as a halide, a sulphate, etc.

In yet another embodiment the cationic associative polyurethanes is chosen from those of formula (Ia) above in which:

R and R′ both independently represent a hydrophobic group,

X and X′ both independently represent a group comprising a quaternary amine,

n and p are zero, and

L, L′, Y and m have the meaning indicated above.

The number-average molecular mass of the cationic associative polyurethanes can range from 400 to 500,000, for instance from 1,000 to 400,000, such as from 1,000 to 300,000.

The term “hydrophobic group” is understood to mean a radical or polymer comprising a linear or branched, saturated or unsaturated hydrocarbon-based chain which can contain at least one hetero atom such as P, O, N or S, or a radical comprising a perfluoro or silicone chain. When it denotes a hydrocarbon-based radical, the hydrophobic group comprises at least 10 carbon atoms, for example from 10 to 30 carbon atoms, for instance from 12 to 30 carbon atoms, such as from 18 to 30 carbon atoms.

In one embodiment, the hydrocarbon-based group originates from a monofunctional compound.

By way of non-limiting example, the hydrophobic group can be derived from a fatty alcohol such as stearyl alcohol, dodecyl alcohol or decyl alcohol. It can also denote a hydrocarbon-based polymer such as, for example, polybutadiene.

Where X and/or X′ denote(s) a group comprising a tertiary or quaternary amine, X and/or X′ can be chosen from the formulae below:

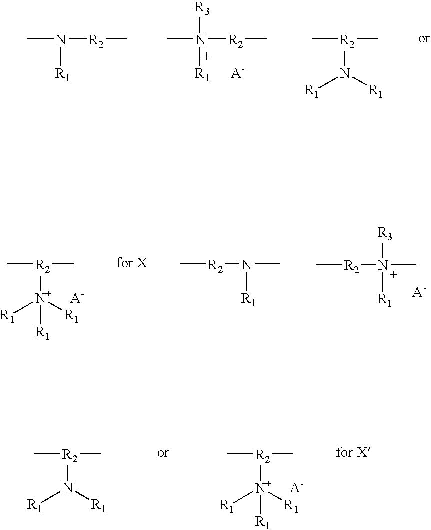

in which:

R₂ is chosen from linear and branched alkylene radicals having from 1 to 20 carbon atoms, which may or may not comprise a saturated or unsaturated ring, or an arylene radical, it being possible for at least one of the carbon atoms to be replaced with a hetero atom chosen from N, S, O and P;

R₁ and R₃, which may be identical or different, are chosen from linear and branched C₁-C₃₀ alkyl and alkenyl radicals, and aryl radicals, it being possible for at least one of the carbon atoms to be replaced with a hetero atom chosen from N, S, O and P;

A⁻ is a physiologically acceptable counterion.

The groups L, L′ and L″ represent a group of formula:

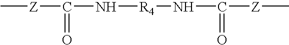

in which:

Z can be chosen from —O— and —S— atoms, and —NH— groups; and

R₄ is chosen from linear and branched alkylene radicals having from 1 to 20 carbon atoms, which may or may not comprise a saturated or unsaturated ring, or an arylene radical, it being possible for at least one of the carbon atoms to be replaced with a hetero atom chosen from N, S, O and P.

The groups P and P′, comprising an amine functional group, can be chosen from at least one of the formulae below:

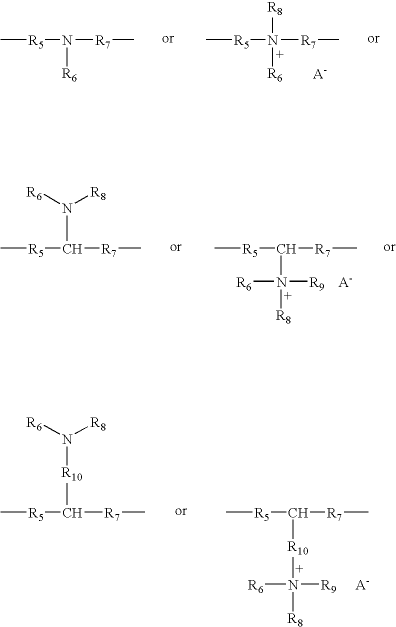

in which:

R₅ and R₇ have the same meanings as R₂ defined above;

R₆, R₈ and R₉ have the same meanings as R₁ and R₃ defined above;

R₁₀ is chosen from optionally unsaturated, linear and branched alkylene groups which may contain at least one hetero atoms chosen from N, O, S and P,

and A⁻ is a physiologically acceptable counterion.

As regards the meaning of Y, the term “hydrophilic group” is intended to mean a polymeric or nonpolymeric water-soluble group.

By way of example, when it is not a polymer, mention may be made of ethylene glycol, diethylene glycol and propylene glycol.

When, in accordance with one embodiment, it is a hydrophilic polymer, mention may, for example, be made of polyethers, sulphonated polyesters, sulphonated polyamides or a mixture of these polymers. The hydrophilic compound can be, for example, a polyether, such as poly(ethylene oxide) or poly(propylene oxide).

The cationic associative polyurethanes of formula (XVIII) that can be used according to the disclosure are formed from diisocyanates and from various compounds having labile hydrogen functional groups. The labile hydrogen functional groups can be alcohol, primary or secondary amine or thiol functional groups giving, after reaction with the diisocyanate functional groups, polyurethanes, polyureas and polythioureas, respectively. The term “polyurethanes” that can be used according to the present disclosure encompasses these three types of polymers, i.e. polyurethanes per se, polyureas and polythioureas, and copolymers thereof.

A first type of compound involved in the preparation of the polyurethane of formula (XVIII) is a compound comprising at least one unit comprising an amine functional group. This compound may be multifunctional, but the compound is preferably difunctional, i.e., according to one embodiment, this compound comprises two labile hydrogen atoms carried, for example, by a hydroxyl, primary amine, secondary amine or thiol functional group. A mixture of multifunctional and difunctional compounds, in which the percentage of multifunctional compounds is low, can also be used.

As indicated above, this compound may comprise at least one unit comprising an amine functional group. It is then a polymer carrying a repetition of the unit comprising an amine functional group.

Compounds of this type can be represented by one of the formulae below:
HZ-(P)n-ZH,
or
HZ-(P′)p-ZH

in which Z, P, P′, n and p are as defined above.

By way of example of a compound comprising an amine functional group, mention may be made of N-methyldiethanolamine, N-tert-butyidiethanolamine and N-sulphoethyldiethanolamine.

The second compound involved in the preparation of the polyurethane of formula (XVIII) is a diisocyanate corresponding to the formula:
O═C═N—R₄—N═C=O

in which R₄ is defined above.

By way of example, mention may be made of methylenediphenyl diisocyanate, methylenecyclohexane diisocyanate, isophorone diisocyanate, toluene diisocyanate, naphthalene diisocyanate, butane diisocyanate and hexane diisocyanate.

A third compound involved in the preparation of the polyurethane of formula (XVIII) is a hydrophobic compound intended to form the terminal hydrophobic groups of the polymer of formula (XVIII).

This compound consists of a hydrophobic group and of a functional group containing a labile hydrogen, for example a hydroxyl, primary or secondary amine, or thiol functional group.

By way of example, this compound may be a fatty alcohol, such as, for instance, stearyl alcohol, dodecyl alcohol or decyl alcohol. When this compound comprises a polymeric chain, it may, for example, be alpha-hydroxyl hydrogenated polybutadiene.

The hydrophobic group of the polyurethane of formula (XVIII) may also result from the quaternization reaction of the tertiary amine of the compound comprising at least one tertiary amine unit. Thus, the hydrophobic group is introduced by the quaternizing agent. This quaternizing agent is a compound of RQ or R′Q type, in which R and R′ are as defined above and Q denotes a leaving group such as a halide, a sulphate, etc.

The cationic associative polyurethane can also comprise a hydrophilic block. This block is provided by a fourth type of compound involved in the preparation of the polymer. This compound may be multifunctional, and in at least one embodiment is difunctional. It is also possible to have a mixture in which the percentage of multifunctional compound is low.

The functional groups having a labile hydrogen are alcohol, primary or secondary amine, or thiol functional groups. This compound may be a polymer terminated at the chain ends with one of these functional groups having a labile hydrogen.

By way of example, when it is not a polymer, mention may be made of ethylene glycol, diethylene glycol and propylene glycol.

When it is a hydrophilic polymer, mention may be made, by way of example, of polyethers, sulphonated polyesters, sulphonated polyamides, or a mixture of these polymers. In one embodiment the hydrophilic compound is a polyether, such as a poly(ethylene oxide) or poly(propylene oxide).

The hydrophilic group marked Y in formula (XVIII) is optional. Specifically, the units comprising a quaternary or protonated amine functional group may suffice to provide the solubility or the water-dispersibility required for this type of polymer in an aqueous solution.

Although the presence of a hydrophilic group Y is optional, in one embodiment, the cationic associative polyurethanes comprise such a group;

(II) derivatives of quaternized cellulose and polyacrylates comprising noncyclic amino side groups.

The derivatives of quaternized cellulose are, for example:

quaternized celluloses modified with groups comprising at least one fatty chain, such as alkyl, arylalkyl or alkylaryl groups containing at least 8 carbon atoms, or mixtures thereof,

quaternized hydroxyethylcelluloses modified with groups comprising at least one fatty chain, such as alkyl, arylalkyl or alkylaryl groups containing at least 8 carbon atoms, or mixtures thereof.

The alkyl radicals carried by the quaternized celluloses or hydroxyethylcelluloses above can contain, for example, from 8 to 30 carbon atoms. The aryl radicals preferably denote phenyl, benzyl, naphthyl or anthryl groups.

As examples of quaternized alkylhydroxyethylcelluloses comprising C₈-C₃₀ fatty chains, the following may be used: the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Amerchol and the products Crodacel QM®, Crodacel QL® (C₁₂ alkyl) and Crodacel QS® (C₁₈ alkyl) sold by the company Croda.

Amphoteric Associative Polymers

The amphoteric associative polymers can be chosen from those comprising at least one noncyclic cationic unit. For instance, those prepared from or comprising 1 to 20 mol % of monomer comprising a fatty chain, for example from 1.5 to 15 mol %, such as 1.5 to 6 mol %, relative to the total number of moles of monomers.

The amphoteric associative polymers that can be used according to one embodiment of the disclosure, for example, comprise, or are prepared by copolymerizing:

1) at least one monomer of formula (XIX) or (XX):

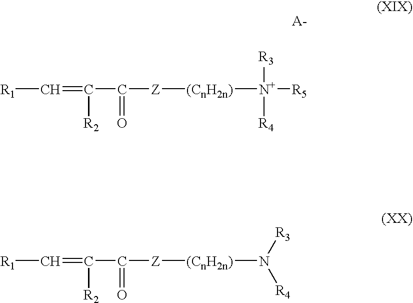

in which R₁ and R₂, which may be identical or different, represent a hydrogen atom or a methyl radical, R₃, R₄ and R₅, which may be identical or different, represent a linear or branched alkyl radical having from 1 to 30 carbon atoms,

Z represents an NH group or an oxygen atom,

n is an integer of from 2 to 5,

A⁻ is an anion derived from an organic or inorganic acid, such as a methosulphate anion, or a halide such as chloride or bromide;

2) at least one monomer of formula (XXI)
R₆—CH═CR₇—COOH (XXI)

in which, R₆ and R₇, which may be identical or different, represent a hydrogen atom or a methyl radical; and

3) at least one monomer of formula (XXII):
R₆—CH═CR₇—COXR₈ (XXII)

in which R₆ and R₇, which may be identical or different, represent a hydrogen atom or a methyl radical, X denotes an oxygen or nitrogen atom, and R₈ denotes a linear or branched alkyl radical having from 1 to 30 carbon atoms;

at least one of the monomers of formula (XIX), (XX) or (XXII) comprising at least one fatty chain.

The monomers of formulae (XIX) and (XX) of the present disclosure can be, for example chosen from:

dimethylaminoethyl methacrylate, dimethylaminoethyl acrylate,
diethylaminoethyl methacrylate, diethylaminoethyl acrylate,

dimethylaminopropyl methacrylate, dimethylaminopropyl acrylate,
dimethylaminopropylmethacrylamide, dimethylaminopropylacrylamide, these monomers being optionally quaternized, for example with a C₁-C₄ alkyl halide or a C₁-C₄ dialkyl sulphate.

For example, the monomer of formula (XIX) can chosen from acrylamidopropyltrimethylammonium chloride and methacrylamidopropyltrimethylammonium chloride.

The monomers of formula (XXI) of the present disclosure can be chosen from acrylic acid, methacrylic acid, crotonic acid and 2-methylcrotonic acid. In one embodiment, the monomer of formula (XXI) is acrylic acid.

The monomers of formula (XXII) of the present disclosure can be, for example, chosen from C₁₂-C₂₂, such as C₁₆-C₁₈, alkyl acrylates or methacrylates.

The monomers constituting the fatty-chain amphoteric polymers of the disclosure can be already neutralized and/or quaternized.

In one embodiment, for example, the ratio of the number of cationic charges/anionic charges is equal to approximately 1.

The amphoteric associative polymers according to the disclosure can comprise from 1 to 10 mol % of the monomer comprising a fatty chain (monomer of formula (XIX), (XX) or (XXII)), such as from 1.5 to 6 mol %.

The weight-average molecular weights of the amphoteric associative polymers according to the disclosure can range from 500 to 50,000,000, such as from 10,000 to 5,000,000.

The amphoteric associative polymers according to the disclosure can also contain other monomers, such as non-ionic monomers, and in particular such as C₁-C₄ alkyl acrylates or methacrylates.

Amphoteric associative polymers according to the disclosure are, for example, described and prepared in International Patent Application Publication No. WO 98/44012.

Among the amphoteric associative polymers according to the disclosure, acrylic acid/(meth)acrylamidopropyltrimethylammonium chloride/stearyl methacrylate terpolymers are used in one embodiment.

The associative polymers of non-ionic type that can be used according to the disclosure can be, for example, chosen from:

(1) celluloses modified with groups comprising at least one fatty chain;

by way of example, non-limiting mention may be made of:
hydroxyethylcelluloses modified with groups comprising at least one fatty chain, such as alkyl, arylalkyl or alkylaryl groups, or mixtures thereof, and in which the alkyl groups are for example, C₈-C₂₂, such as the product Natrosol Plus Grade 330 CS® (C₁₆ alkyl) sold by the company Aqualon, or the product Bermocoll EHM 1 00® sold by the company Berol Nobel,
those modified with alkylphenol polyalkylene glycol ether groups, such as the product Amercell Polymer HM-1500® (nonylphenol polyethylene glycol (15) ether) sold by the company Amerchol;

(2) hydroxypropyl guars modified with groups comprising at least one fatty chain, such as the product Esaflor HM 22® (C₂₂ alkyl chain) sold by the company Lamberti, or the products RE210-18® (C₁₄ alkyl chain) and RE205-1® (C₂₀ alkyl chain) sold by the company Rhone Poulenc;

(3) copolymers of vinylpyrrolidone and of fatty-chain hydrophobic monomers, of which mention may, for example, be made of:
the products Antaron V216® or Ganex V216® (vinylpyrrolidone/hexadecene copolymer) sold by the company I.S.P.,
the products Antaron V220® or Ganex V220® (vinylpyrrolidone/eicosene copolymer) sold by the company I.S.P.;

(4) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®;

(5) copolymers of hydrophilic methacrylates or acrylates and of hydrophobic monomers comprising at least one fatty chain, such as, for example, the polyethylene glycol methacrylate/lauryl methacrylate copolymer;

(6) polyurethane polyethers comprising, in their chain, both hydrophilic blocks that are most commonly polyoxyethylenated in nature and hydrophobic blocks that may be aliphatic sequences alone and/or cycloaliphatic and/or aromatic sequences;

(7) polymers with an aminoplast ether skeleton containing at least one fatty chain, such as the compounds Pure Thix® proposed by the company Sud-Chemie.

For example, the polyether polyurethanes comprise at least two lipophilic hydrocarbon-based chains having from 6 to 30 carbon atoms, separated by a hydrophilic block, it being possible for the hydrocarbon-based chains to be pendent chains or chains at the end of a hydrophilic block. For instance, it is possible for at least one pendent chain to be envisaged. Furthermore, the polymer may comprise a hydrocarbon-based chain at one end or at both ends of a hydrophilic block.

The polyether polyurethanes may be multiblock, such as triblock form. The hydrophobic blocks may be at each end of the chain (for example: triblock copolymer with a hydrophilic central block) or distributed both at the ends and in the chain (multiblock copolymer, for example). These same polymers can also be grafted polymers or star polymers.

The non-ionic polyether polyurethanes comprising a fatty chain can be triblock copolymers in which the hydrophilic block is a polyoxyethylenated chain containing from 50 to 1,000 oxyethylenated groups. The non-ionic polyether polyurethanes contain a urethane bond between the hydrophilic blocks, hence the origin of the name.

By extension, also among the non-ionic polyether polyurethanes comprising a fatty chain are those in which the hydrophilic blocks are linked to the lipophilic blocks by other chemical bonds.

By way of examples of non-ionic polyether polyurethanes comprising a fatty chain that can be used in the invention, use may also be made of Rheolate 205® containing a urea function, sold by the company Rheox, or Rheolate® 208, 204 or 212, and also Acrysol RM 184®.

Mention may also be made of the product Elfacos T210® comprising a C₁₂-14 alkyl chain and the product Elfacos T212® comprising a C₁₈ alkyl chain, from Akzo.

The Rohm & Haas product DW 1206B® comprising a C₂₀ alkyl chain and a urethane bond, provided at 20% solids content in water, can also be used.

Solutions or dispersions of these polymers, such as in water or in an aqueous/alcoholic medium, can also be used. By way of example of such polymers, mention may be made of Rheolate® 255, Rheolate® 278 and Rheolate® 244 sold by the company Rheox. The product DW 1206F and DW 1206J proposed by the company Rohm & Haas can also be used.

The polyether polyurethanes that can be used according to the disclosure can be, for example, those described in the article by G. Formum, J. Bakke and Fk. Hansen-Colloid Polym. Sci 271, 380.389 (1993).

For further example, use can be made of a polyether polyurethane that can be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyether polyurethanes are sold for example, by the company Rohm & Haas under the names Aculyn 46® and Aculyn 44®. Aculyn 46® is a polycondensate of polyethylene glycol comprising 150 or 180 mol of ethylene oxide, of stearyl alcohol and of methylenebis(4-cyclohexyl isocyanate) (SMDI), at 15% by weight in a maltodextrin (4%) and water (81%) matrix; Aculyn 44® is a polycondensate of polyethylene glycol comprising 150 or 180 mol of ethylene oxide, or decyl alcohol and of methylenebis(4-cyclohexyl isocyanate) (SMDI), at 35% by weight in a propylene glycol (39%) and water (26%) mixture].

The composition can also contain fillers.

In the present disclosure, the term “fillers” is understood to mean colorless or white, mineral or synthetic, lamellar or nonlamellar particles intended to give the composition body or rigidity and/or to confer softness, a matt aspect and uniformity on the makeup. The fillers that can be used in the cosmetic compositions of the present disclosure are chosen, for example, from talc, mica, silica, kaolin, nylon powder and polyethylene powder, Teflon®, starch, boron nitride, polymer microspheres such as Expancel® from the company Nobel Industrie or Polytrap® from the company Dow Corning, silicone resin microbeads such as Tospearls® from the company Toshiba, precipitated calcium carbonate, magnesium carbonate or hydrocarbonate, and metal soaps derived from C₈-C₂₂ carboxylic acids.

The fillers can be present in an amount ranging from 0 to 80% by weight, for instance from 5 to 15% by weight, relative to the final weight of the cosmetic composition.

The appropriate cosmetic medium for the cosmetic compositions can comprise at least one solvent chosen from water, ketones such as methyl ethyl ketone, methyl isobutyl ketone, diisobutyl ketone, isophorone, cyclohexanone or acetone, lower alcohols such as ethanol, isopropanol, diacetone alcohol, 2-butoxyethanol or cyclohexanol, polyols such as propylene glycol or pentylene glycol or polyethylene glycols, alkylene glycol ethers, such as propylene glycol monomethyl ether, the acetate of propylene glycol monomethyl ether or dipropylene glycol ether, C₂-C₇ alkyl acetates, such as methyl acetate, ethyl acetate, propyl acetate, butyl acetate or isopentyl acetate, ethers such as diethyl ether, dimethyl ether or dichlorodiethyl ether and volatile oils such as cyclic or linear volatile silicone oils, synthetic hydrocarbon-based volatile oils or fluoro oils, or mixtures thereof.

These liquids that are solvent in nature can be present in an amount ranging from 1 and 40% by weight, relative to the total weight of the dye composition, such as from 5 and 30% by weight.

The composition may comprise at least one adjuvant normally used in the cosmetics field, such as antioxidants, fragrances, preserving agents, lipophilic or hydrophilic cosmetic active agents, moisturizers, vitamins, essential fatty acids, sphingolipids, self-tanning agents, sunscreens, antifoams, free-radical scavengers, anionic, cationic, non-ionic, amphoteric or zwitterionic polymers, or mixtures thereof, other than the thickening polymers described above, penetrating agents, sequestering agents, buffers, dispersing agents, conditioning agents other than those mentioned above, cationic polymers, chitosans and derivatives, ceramides, preserving agents, amino acids such as arginine, cysteine, glycine or taurine, or opacifiers.

These above adjuvant can be present in an amount, for each of them, ranging from 0.01 to 20% by weight, relative to the total weight of the composition.

For example, the compositions of the present disclosure contain at least one surfactant and/or at least one thickener.

Of course, those skilled in the art would take care to select the optional additional compounds in such a way that the beneficial properties of the composition according to the disclosure are not, or are virtually not, impaired by the envisaged addition.

The pH of the dye composition in accordance with the disclosure is can range from 3 to 12, such as from 5 to 11.

It can be adjusted to the desired value by means of acidifying or basifying agents normally used in coloration, or alternatively by means of conventional buffer systems.

Among acidifying agents, mention may be made, by way of example, of inorganic or organic acids, such as hydrochloric acid, orthophosphoric acid or sulphuric acid, carboxylic acids such as acetic acid, tartaric acid, citric acid or lactic acid, and sulphonic acids.

Among basifying agents, mention may be made, by way of example, of aqueous ammonia, alkali metal carbonates, alkanolamines such as mono-, di- and triethanolamines, and derivatives thereof, sodium hydroxide or potassium hydroxide, and compounds of formula (V) below:

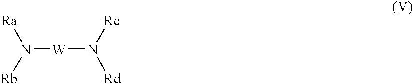

in which W is a propylene residue optionally substituted with a hydroxyl group or a C₁-C₄ alkyl radical; Ra, Rb, Rc and Rd, which may be identical or different, represent a hydrogen atom, a C₁-C₄ alkyl radical or a C₁-C₄ hydroxyalkyl radical.

The cosmetic compositions of the present disclosure may be in any form usually encountered in the cosmetics field, i.e. in the form of a lotion, a suspension, a dispersion, an organic, aqueous or aqueous-alcoholic solution optionally thickened or gelled, a foam, a spray, an oil-in-water, water-in-oil or multiple emulsion, a loose, compact or cast powder, an anhydrous solid or paste, or a cream.

In one embodiment it can be a care, hygiene and/or makeup product. Other embodiments of the cosmetic compositions of the present disclosure are represented by hair compositions such as a dye composition, nail varnishes and makeup compositions for the face, the body or the integuments (nails, eyelashes, eyebrows, hair), such as an eyeshadow, blusher, eyeliner, mascara, loose or compact powder, foundation, tinted cream, lipstick, concealer stick, etc.

The present disclosure also relates to a process for changing the coloration or shade of the skin, keratin fibers, lips or integuments using the composition described above. This process comprises applying to the skin, the lips or the integuments, a composition as disclosed herein, optionally in the presence of an oxidizing agent, and then subjecting the lips or the integuments to a mechanical stress, such as a manual friction, so as to obtain the change in coloration or shade desired. The rubbing time and force depend on the desired shade.

The present disclosure also relates to a device with several compartments or “kit”, such as a 2-compartment device, for dyeing keratin fibers, including the hair, in which at least one first compartment contains the dye composition of the present disclosure and at least one second compartment contains at least one cosmetic adjuvant as defined above. This device may be equipped with a means for delivering the desired mixture onto the hair, such as the devices described in French Patent No. FR-2 586 913.

The present disclosure also relates to the use of the composition as defined above in hair dyeing and for makeup for the skin, the lips or the integuments, for example for allowing changes in tints.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

The examples which follow illustrate the invention without limiting the scope thereof.

### EXAMPLES

### Example 1

The dye of formula (IV) was incorporated into the following formulation:

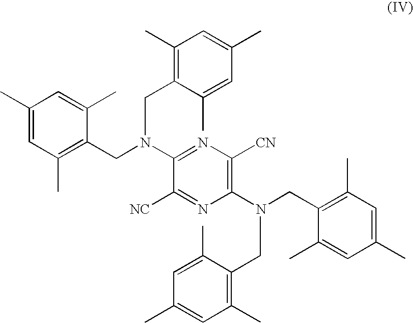

The following formulation was prepared:

| Compound | Amount | |
|---|---|---|
| Dye of formula (IV) | 10⁻³ | mol % |
| Cyclopentasiloxane | Qs 100 | g |

The formulation was applied to the skin, the lips and the integuments, and then the volatile silicone was allowed to evaporate. At this stage, a yellow coloration was obtained, which, by simple mechanical rubbing, became orange.

### Example 2

The dye of formula (V) is incorporated into the following formulation:

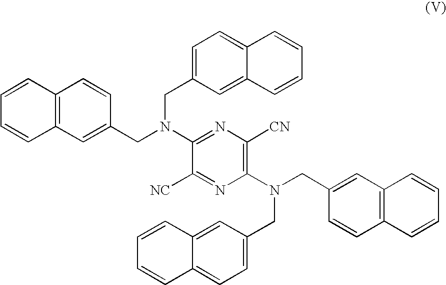

The following formulation was prepared:

| Compound | Amount | |
|---|---|---|
| Dye of formula (V) | 10⁻³ | mol % |
| Cyclopentasiloxane | Qs 100 | g |

The formulation was applied to the skin, the lips and the integuments, and the volatile silicone was then allowed to evaporate. At this stage, a yellow coloration was obtained, which, by simple mechanical rubbing, became orange.

### Example 3

The dye of formula (VI) was incorporated into the following formulation:

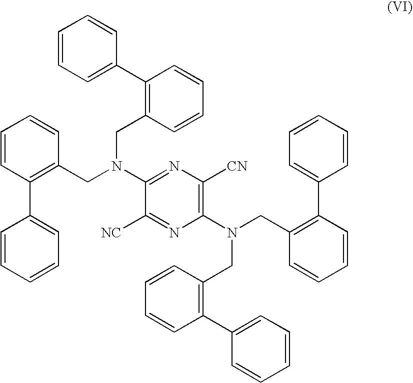

The following formulation was prepared:

| Compound | Amount | |
|---|---|---|
| Dye of formula (VI) | 10⁻³ | mol % |
| Cyclopentasiloxane | Qs 100 | g |

The formulation was applied to the skin, the lips and the integuments, and the volatile silicone was then allowed to evaporate. At this stage, a yellow coloration was obtained, which, by simple mechanical rubbing, became orange.

### Example 4

The dye of formula (VII) was incorporated into the following formulation:

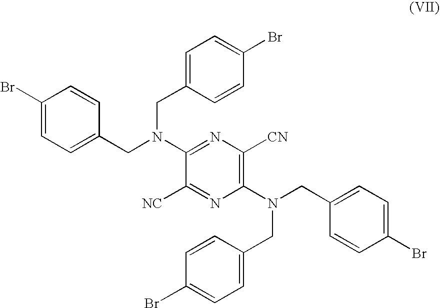

The following formulation was prepared:

| Compound | Amount | |
|---|---|---|
| Dye of formula (VII) | 10⁻³ | mol % |
| Cyclopentasiloxane | Qs 100 | g |

The formulation was applied to the skin, the lips and the integuments, and the volatile silicone was then allowed to evaporate. At this stage, a yellow coloration was obtained, which, by simple mechanical rubbing, became orange.

### Example 5

The dye of formula (VIII) was incorporated into the following formulation:

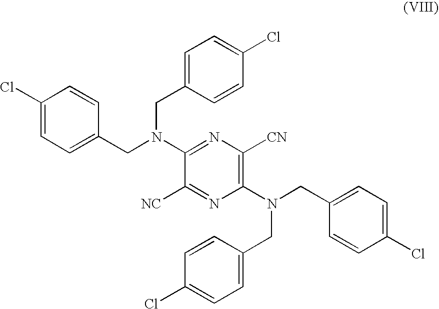

The following formulation was prepared:

| Compound | Amount | |
|---|---|---|
| Dye of formula (VIII) | 10⁻³ | mol % |
| Cyclopentasiloxane | Qs 100 | g |

The formulation was applied to the skin, the lips and the integuments, and the volatile silicone was then allowed to evaporate. At this stage, a yellow coloration was obtained, which, by simple mechanical rubbing, became orange.

### Example 6

The following formulation was prepared:

| Compound | Amount | |
|---|---|---|
| Dye of formula (IV) | 10⁻² | mol % |
| Benzyl alcohol | 5 | g |
| Ethanol | 20 | g |
| Lauryl ether sulphate comprising | 2 | g AM |
| 2 mol of EO | | |
| Water | Qs 100 | g |

The formulation was applied to the hair. After drying, a yellow coloration was obtained, which became orange by simple mechanical rubbing.

What is claimed is:

1. A cosmetic composition comprising, in an appropriate cosmetic medium, at least one tribochromic compound.

2. The cosmetic composition according to claim 1, further comprising at least one compound chosen from direct dyes, oxidation dyes, surfactants, thickeners, oils, waxes, gums, pigments and pearlescent agents.

3. The cosmetic composition according to claim 1, wherein the at least one tribochromic compound is chosen from those of formulae (I)-(III): 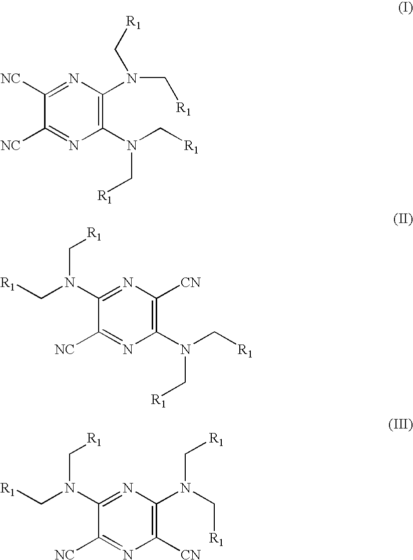
wherein
R₁, which may be identical or different, is chosen from condensed and noncondensed C₆-C₃₀ aryl radicals optionally substituted with at least one entity chosen from halogen atoms, and C₁-C₁₀ alkyl, hydroxyl, (C₁-C₁₀)alkoxyamino, (C₁-C₁₀)mono and dialkylamino, mono and dihydroxy(C₁-C₁₀)alkylamino, (C₁-C₁₀)alkylhydroxy(C₁-C₁₀)alkylamino, mono and polyhydroxy(C₁-C₁₀)alkyl, C₆-C₃₀ aryl, carboxyl, (C₁-C₁₀)alkoxycarbonyl, sulpho, C₂-C₁₀ acyl, (C₁-C₁₀) acyloxy, aminocarbonyl, nitro, cyano and ureido groups, it being possible for R₁ to be substituted with an electron-withdrawing group or with an electron-donor group.

4. The cosmetic composition according to claim 3, wherein the substituents R₁ are identical.

5. The cosmetic composition according to claim 4, wherein the substituents R₁ are identical and are chosen from trimethylphenyl, biphenyl, naphthalenylmethyl, chlorophenyl or bromophenyl groups.

6. The cosmetic composition according to claim 3, wherein the electron-withdrawing group is chosen from halogen atoms, nitro groups, cyano groups, and —SO₃⁻ groups.

7. The cosmetic composition according to claim 3, wherein the electron-donor group is chosen from alkyl groups, alkoxy groups, unsubstituted amine groups, amine groups substituted with an alkyl group, and aryl groups.

8. The cosmetic composition according to claim 3, wherein the at least one tribochromic compound is chosen from those of formulae (IV)-(VIII):

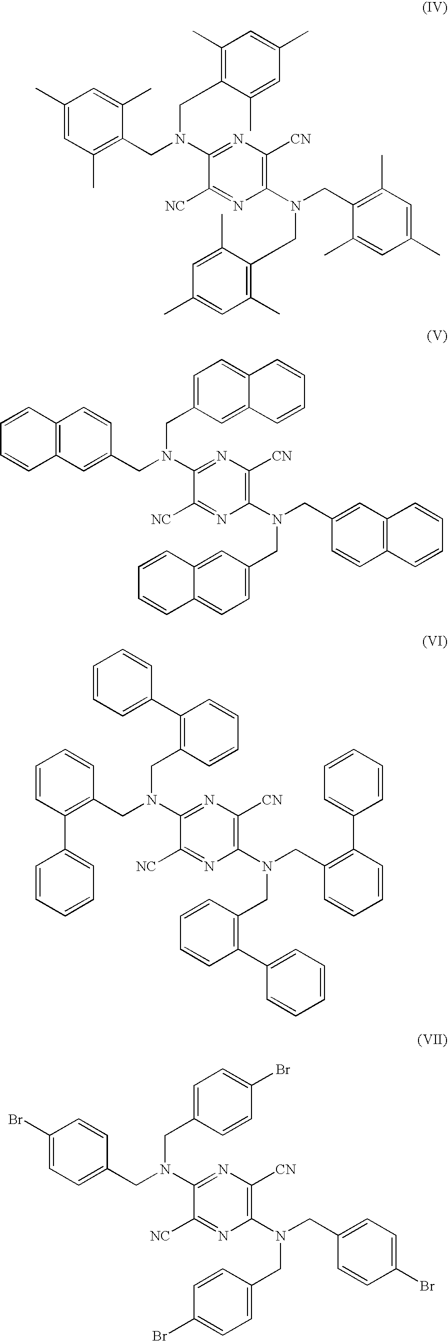

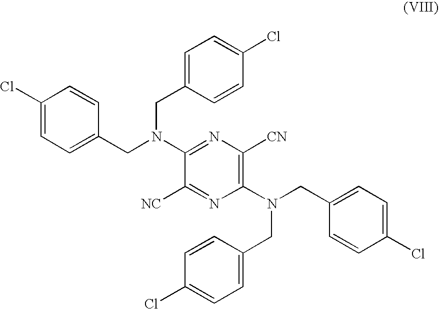

9. The cosmetic composition according to claim 1, wherein the at least one tribochromic compound is present in an amount ranging 0.0001% to 30% by weight, relative to the total weight of the composition.

10. The cosmetic composition according to claim 9, wherein the at least one tribochromic compound is present in an amount ranging from 0.01% to 10% by weight, relative to the total weight of the composition.

11. The cosmetic composition according to claim 2, wherein the at least one direct dye is chosen from neutral, acidic and cationic nitrobenzene direct dyes; neutral, acidic and cationic azo direct dyes; neutral, acidic and cationic quinone direct dyes; azine direct dyes; triarylmethane direct dyes; indoamine direct dyes; and natural direct dyes.

12. The cosmetic composition according to claim 11, wherein the at least one direct dye is present in an amount ranging from 0.001% to 20% by weight, relative to the total weight of the composition.

13. The cosmetic composition according to claim 12, wherein the at least one direct dye is present in an amount ranging from 0.005% to 10% by weight, relative to the total weight of the composition.

14. The cosmetic composition according to claim 2, wherein the at least one oxidation base is chosen from para-phenylenediamines, bisphenylalkylenediamines, para-aminophenols, ortho-aminophenols, heterocyclic bases, and addition salts thereof.

15. The cosmetic composition according to claim 2, wherein the at least one coupler is chosen from meta-phenylenediamine couplers, meta-aminophenol couplers, meta-diphenol couplers, naphthalene couplers, heterocyclic couplers, and addition salts thereof.

16. The cosmetic composition according to claim 14, wherein the at least one oxidation base is present in an amount, for each base, ranging from 0.001% to 10% by weight, relative to the total weight of the dye composition.

17. The cosmetic composition according to claim 16, wherein the at least one oxidation base is present in an amount, for each base, ranging from 0.005% to 6% by weight, relative to the total weight of the dye composition.

18. The cosmetic composition according to claim 15, wherein the at least one coupler is present in an amount, for each coupler, ranging from 0.001% to 10% by weight, relative to the total weight of the dye composition.

19. The cosmetic composition according to claim 18, wherein the at least one coupler is present in an amount, for each coupler, ranging from 0.005% to 6% by weight, relative to the total weight of the dye composition.

20. The cosmetic composition according to claim 14, further comprising at least one oxidizing agent.

21. The cosemetic composition according to claim 15, further comprising at least one oxidizing agent.

22. The cosmetic composition according to claim 2, further comprising a fatty phase comprising at least one oils or wax at ambient temperature, chosen from those of animal, plant, mineral and synthetic origin.

23. The cosmetic composition according to claim 22, wherein the at least one oil is a volatile oil.

24. The cosmetic composition according to claim 2, wherein the at least one thickener is chosen from mineral and organic thickeners.

25. The cosmetic composition according to claim 24, wherein the at least one organic thickener is a polymer.

26. The cosmetic composition according to claim 2, wherein the at least one surfactant is chosen from anionic, non-ionic, amphoteric and cationic surfactants.

27. The cosmetic composition according to claim 1, wherein the appropriate cosmetic medium for cosmetic compositions comprises at least one adjuvant chosen from antioxidants, fragrances, preserving agents, lipophilic and hydrophilic cosmetic active agents, moisturizers, vitamins, essential fatty acids, sphingolipids, self-tanning agents, sunscreens, antifoams, free-radical scavengers, anionic, cationic, non-ionic, amphoteric and zwitterionic polymers, penetrating agents, sequestering agents, buffers, dispersing agents, conditioning agents, cationic polymers, chitosans and derivatives, ceramides, preserving agents, amino acids, and opacifiers.

28. The cosmetic composition according to claim 1, wherein the appropriate cosmetic medium comprises at least one solvent chosen from water, ketones, alcohols, polyols, alkylene glycol ethers, C₂-C₇ alkyl acetates, ethers and aldehydes.

29. The cosmetic composition according to claim 1, wherein it is in the form of a lotion, a suspension, a dispersion, an organic, aqueous or aqueous-alcoholic solution optionally thickened or gelled, a foam, a spray, an oil-in-water, water-in-oil or multiple emulsion, a loose, compact or cast powder, an anhydrous solid or paste, or a cream.

30. The cosmetic composition according to claim 1, wherein it is a dye composition for keratin fibers.

31. The cosmetic composition according to claim 1, wherein it is a nail varnish.

32. The cosmetic composition according to claim 1, wherein it is a makeup composition.

33. A process for changing the coloration or shade of the skin, keratin fibers, lips or integuments, comprising
applying to the skin, keratin fibers, lips or integuments a composition comprising, in an appropriate cosmetic medium, at least one tribochromic compound, and
subjecting the skin, keratin fibers, lips or integuments to a mechanical stress, so as to obtain the change in coloration or shade desired, wherein the mechanical stress comprises rubbing the composition onto the skin, keratin fibers, lips, or integuments, for a time and with a force that vary depending on the desired shade or coloration.

34. The process according to claim 33, wherein the mechanical stress is a manual friction.

35. The process according to claim 33, wherein the mechanical stress comprises a rubbing force that ranges from 0.01 N to 50 N.

36. The process according to claim 35, wherein the mechanical stress comprises a rubbing force that ranges from 1 N to 20 N.

37. The process according to claim 35, wherein said process for changing the coloration or shade of the skin, keratin fibers, lips or integuments is chosen from
making up the skin, lips, or integuments, and
dyeing the keratin fibers.

38. A multi-compartment kit, for dyeing human keratin fibers, comprising
at least one first compartment that contains a composition comprising, in an appropriate cosmetic medium, at least one tribochromic compound; and
at least one second compartment that contains at least one cosmetic adjuvant.
